(19) United States
(12) Patent Application Publication
(10) Pub. No.: US 2011/0087098 A1
(43) Pub. Date: Apr. 14, 2011

(54) MAMMOGRAPHY SYSTEM AND METHOD FOR SONOGRAPHIC AND RADIOGRAPHIC EXAMINATION OF A BREAST

(75) Inventors: Daniel Fischer, Erlangen (DE); Thomas Mertelmeier, Erlangen (DE)

(73) Assignee: SIEMENS AKTIENGESELLSCHAFT, München (DE)

(21) Appl. No.: 12/918,562

(22) PCT Filed: Jan. 21, 2009
(86) PCT No.: PCT/EP2009/050624

§ 371 Date: Aug. 20, 2010

(30) Foreign Application Priority Data
Feb. 20, 2008 (DE) 10 2008 009 967.8

(51) Int. Cl. A61B 8/00

(52) U.S. Cl. 600/439

(57) ABSTRACT

A mammography device for examining a breast has a support plate and a compression plate, between which the breast is positioned and compressed during a radiographic examination. An ultrasound transducer for obtaining a sonogram of the breast is brought into direct contact with the breast either by temporary mounting in one of the support plate or the compression plate or by exchanging one of the compression plate or the support plate with another plate that contains the ultrasound transducer.

### BACKGROUND OF THE INVENTION

1. Field of the Invention

The invention concerns a mammography system and a method for sonographic and radiographic examination of a breast.

2. Description of the Prior Art

A combined system for implementing a radiographic as well as a sonographic examination of a breast is known from U.S. Pat. No. 6,574,499.

Examinations of the breast—in particular the female breast—are in many cases implemented with the goal of detecting tumors at as early a stage as possible. Via continuous improvement of the imaging methods it is sought to generate images with high significance in order to be able to differentiate benign variations from malignant variations with high certainty. One goal in the improvement of the existing devices and methods is to reduce the number of incorrect findings, i.e. to reduce the number of suspicious findings that are not caused by malignant variations and the number of undetected tumors.

Mammography is a widespread method for examination of the breast. In a mammographic examination, the breast is positioned and compressed between a support plate and a compression plate in a mammography system. A two-dimensional x-ray image of the compressed breast is subsequently generated. Tomosynthesis is a further development of mammography. In this modality, a number of 2D projection images are acquired by exposure of the breast from different directions. The 2D projection images are calculated into a tomosynthetic 3D x-ray image. In contrast to conventional mammography images, a tomosynthetic 3D x-ray image consists of not only one individual projection image of the breast; the examination subject is displayed in a plurality of parallel slice planes or, respectively, slices. A tomosynthetic 3D x-ray image thus contains depth information.

It is known that mammography loses sensitivity in dense breast tissue (as occurs primarily in young women), such that there is a higher risk that lesions or calcifications indicating carcinomas are not detected. For example, in women over 50 years of age the sensitivity of the mammography is approximately 70% to 95% but in young women with dense breast tissue it goes below 50% (Warren Burhenne, L J et al. Radiology 215, 554-562, 2000).

Sonography implemented with the use of ultrasonic waves is an alternative method for the examination of the breast. Information that is complementary to the mammogram can be acquired with the use of sonography, such that tumors can be found that would remain undetected by a mammography examination (Kolb T M, Radiology 225, 165-175, 2000).

It is known that mammography and sonography are implemented with different postures of the patient to be examined. While mammography is typically implemented with a compressed breast at a standing patient, sonographic examination typically ensues on a recumbent patient with an uncompressed breast. In mammography the compressed breast is exposed in a direction that is predetermined by the mammography apparatus. In sonography, the examination ensues with a hand-held ultrasound head. The images acquired with the aid of mammography and with the aid of sonography are therefore difficult to compare with one another. A geometric association of subjects visible in both images is therefore possible with difficulty.

Prototypes of integrated apparatuses to implement sonographic and radiographic examinations are known in which it is sought to make an association between a sonographic image and a radiographic image. For example, such a device is known from U.S. Pat. No. 6,574,499 (cited above).

Such devices provide an ultrasonic examination of a breast compressed between the compression plates of a mammography system. The ultrasound head is thereby directed in a wandering scanning motion over the top side of a compression plate. The radiated ultrasound signals must consequently be injected through the compression plate into the breast to be examined. The injection of the ultrasound through the compression plate is technically problematic since the boundary surfaces of the compression plate are additionally located between the ultrasound source and the examination subject. Moreover, the ultrasound head must be directed in a (possibly wandering) scanning motion across the breast, which leads to relatively long examination times. The ultrasound device arranged above the compression plate can moreover hinder the x-ray examination (mammography).

### SUMMARY OF THE INVENTION

An object of the present invention to provide a method and a mammography system for sonographic and radiographic examination of a breast with which an association of sonographic and radiographic image data is possible. Moreover, the sonographic examination should be improved with regard to the examination speed and the achievable image quality.

The method according to the invention for the examination of a breast combines a radiographic examination and a sonographic examination. It is not significant in what order the examinations are implemented. Within the scope of the radiographic examination, the breast is positioned on a support plate of a mammography system and is compressed between this plate and an opposite compression plate. The breast is subsequently exposed with radiation in the compressed state to acquire a mammography image. Conventionally, after a radiographic examination of the breast has occurred, the compression plate is raised relative to the support plate. Within the scope of the sonographic examination, the breast is compressed between a flat (thus two-dimensionally extended) ultrasonic transducer on the one side and the compression plate or support plate on the other side in the mammography system, and is thereby brought into direct contact with the ultrasonic transducer. The ultrasound transducer has a number of ultrasound transducer elements that are advantageously arranged in a laminar matrix. A sonography image of the compressed breast is subsequently acquired with the aid of the ultrasonic transducer.

The method according to the invention in particular has the following advantages. At the beginning of the examination the breast is positioned on a support plate. The position of the breast that is essentially fixed in this manner is now maintained for both the mammography examination and the sonographic examination. In other words: the breast is located at nearly the same location in both examinations. Moreover, the breast is advantageously compressed in a similar manner for both the acquisition of the mammogram and the acquisition of the sonogram. As a result of the very similar positioning, the mammogram and the sonogram are easily comparable with one another.

An additional important advantage of the method according to the invention lies in the use of a large-area ultrasound transducer. The ultrasound transducer is directly brought into contact with the breast to be examined; the coupling is advantageously assisted via a generally known coupling medium. No additional boundary surface—as is the case in the injection via a compression plate, for example—must be overcome in the injection of the ultrasound waves into the breast. Moreover, the large-area ultrasound transducer allows a quick examination of the breast since in particular a scanning motion of the ultrasound transducer must implemented.

As is mentioned, it is possible to implement the radiographic examination of the breast before the sonographic examination. This is primarily useful in the cases when a breast with a high density should be examined, for example within the scope of screening examinations. The density of the breast can be determined using the mammography image. A sonographic examination of the breast is advantageously conducted only in the case in which the density of a predetermined limit value is exceeded. The entire examination method can thus be limited to the necessary partial examinations and can therefore be accelerated.

According to an embodiment, the sonographic examination is implemented before the radiographic examination. Particularly for young patients or patients with (typically) dense breast tissue, a sonographic examination is sometimes more enlightening than a radiographic examination. If a finding is made within the scope of the sonographic examination, a radiographic examination can additionally be used to clarify the finding. The described procedure has the advantage that superfluous x-ray exposure can be avoided precisely with young patients with high x-ray sensitivity.

In another embodiment, the compression plate or the support plate is exchanged with an additional compression plate or support plate. The additional compression plate or support plate has an ultrasound transducer on its side facing the breast. The breast is subsequently compressed between the exchanged, additional compression plate or support plate on the one side and the un-exchanged, opposite support plate or compression plate on the other side. Within the course of the compression, the ultrasound transducer is brought into direct contact with the breast; a sonogram is subsequently acquired.

As an alternative to the described exchange of the compression plate or support plate, an ultrasound transducer is arranged on the side of the compression plate or support plate facing the breast within the scope of the sonographic examination (which normally follows the radiographic examination). The breast is subsequently compressed between the ultrasound transducer on the one side and the opposite support plate or compression plate on the other side. The ultrasound transducer is thereby likewise brought into direct contact with the breast, and a sonogram of the compressed breast is subsequently acquired with the use of the ultrasound transducer. In the event that the sonographic examination is conducted before the radiographic examination, the ultrasound transducer is naturally located at the specified location before the beginning of the sonographic examination. The ultrasound transducer is removed from this location before the radiographic examination is conducted.

According to one embodiment, only the exchange of the compression plate for an additional compression plate containing the ultrasound transducer is provided. Analogously, according to a further embodiment the ultrasound transducer is arranged on side of the compression plate facing the breast. According to the aforementioned embodiments, the breast can advantageously remain positioned on the support plate during the entire examination since this is not exchanged. The sonogram and mammogram are consequently highly comparable with one another since the breast is located in nearly the same position in both examinations. If, according to a method variant, the radiographic examination is conducted before the sonographic examination, the compression plate is exchanged for the additional compression plate containing the ultrasound transducer. If the sonographic examination is conducted before the radiographic examination, the additional compression plate is accordingly, conversely exchanged for the compression plate.

According to a further embodiment, the compression plate or support plate is brought into its position from such a direction that the patient can remain essentially unchanged in his examination position while the compression plate or support plate is being exchanged. The same applies if the ultrasound transducer attached on the side of the compression plate or support plate that faces toward the breast. The compression plate or support plate containing the ultrasound transducer or the ultrasound transducer itself are thus brought into its position from such a direction that that side of the compression plate or support plate that is facing the patient during the radiographic examination is facing away. Since the patient remains in the same posture during the radiographic examination and sonographic examination—thus for the entire duration of the examination—optimally reproducible results can be achieved.

According to a further embodiment, the image data set of the mammogram and that of the sonogram are brought into registration with one another. By registration of the two images it is possible to compare the mammogram and the sonogram directly with one another. Corresponding regions in both images can be identified so that both radiographic and sonographic information can be obtained from a structure identified in the images.

According to a further embodiment, the significance of the images is increased by the radiographic examination being implemented as a tomosynthetic examination. In this tomosynthetic examination a number of different x-ray images are calculated (reconstructed) to form tomosynthetic 3D mammography image. In contrast to conventional mammography images, this image contains information about layers of the subject situated atop one another in the beam direction. An image acquired within the scope of a sonographic examination likewise always contains depth information about the examined subject. Two examination results that contain both depth information of the examined subject are advantageously combined with one another via the combination of the two cited methods. For example, structures that lie one behind the other in the beam direction and can only be differentiated with difficulty with the aid of conventional mammography can be examined with such a combined method. According to a further embodiment, the significance of the information obtained with the aid of such a combined method is increased in that the image data set of the 3D mammogram and the image data set of the sonogram are registered with one another. Such 3D images registered with one another allow the simple association of subjects shown in both images. It is advantageously possible to superimpose the two images.

According to a further advantageous embodiment, the ultrasound transducer is operated at a frequency from approximately 5 MHz to 15 MHz, advantageously at a frequency in the range from 7 MHz to 9 MHz. The aforementioned ultrasound frequencies are particularly suitable for the examination of breast tissue.

A mammography system according to the invention for the examination of a breast has a support plate and a compression plate between which the breast can be positioned and compressed during a radiographic examination. According to a first variant of the mammography system according to the invention, the support plate and/or the compression plate can be exchanged for an additional support plate or compression plate. This additional support plate or compression plate has—in its side facing the breast—an ultrasound transducer to implement a sonographic examination. According to a further variant, the support plate and/or the compression plate has a mount to accommodate an ultrasound transducer. The mount is designed so that an accommodation of the ultrasound transducer on the side of the support plate or compression plate facing the breast is possible.

Significant advantages of the mammography system according to the invention—which is suitable for the implementation of the method according to the invention—are already cited in connection with the method according to the invention.

The device according to the invention can be an existing mammography system which is made suitable (retrofitted) for sonographic examinations by exchanging the compression plate or support plate or by attaching corresponding mounts.

According to a further embodiment, only the compression plate can be exchanged or is provided with corresponding mounts to accommodate the ultrasound transducer. As mentioned in connection with the method according to the invention, in such a mammography system the breast can remain positioned on the support plate during the entire examination, which improves the comparability of the sonographic examination and the radiographic examination.

According to a further embodiment, the mounts present at the mammography system or at the support plate and/or compression plate are designed so that an accommodation of the ultrasound transducer is provided from a direction that is facing away from that side of the device that is facing a patient during the radiographic examination. Such an advantageous embodiment of the mount allows the ultrasound transducer to be placed into the mount without the patient having to deviate from the posture provided for the implementation of the radiographic examination. The same applies for a device in which the compression plate or support plate is exchanged for an additional compression plate or support plate. Here the device can advantageously be designed such that the plates can be exchanged without the patient having to change position.

According to a further advantageous embodiment, the ultrasound transducer is inserted into a side of the compression plate or support plate that faces the breast. Moreover, this advantageously has a number of ultrasound transducer elements which are preferably capacitively activated micro-ultrasound transducer elements. The resolution capability of an ultrasound transducer that is made up of a plurality of ultrasound transducer elements increases proportionally with the number of ultrasound transducer elements. Micro-machined preferably lithographically produced, and capacitively controlled micro-ultrasound transducer elements can be mass-produced at advantageous costs and high quality with production methods known from semiconductor engineering. These ultrasound transducer elements—also designated as capacitive micro-fabricated ultrasound transducers (CMUTs)—have different advantages relative to conventional ultrasound transducer elements. For example, such a CMUT ultrasound transducer—thus an arrangement of many ultrasound transducer elements—is significantly simpler to contact than an arrangement of conventional ultrasound transducer elements. In a CMUT ultrasound transducer, a majority of the wiring has already been conducted in the manufacture of the ultrasound transducer. Moreover, such ultrasound transducer arrays often contain a portion of the transducer and amplifier electronics. In particular, existing mammography systems can be retrofitted with such an ultrasound transducer with limited technical cost.

### BRIEF DESCRIPTION OF THE DRAWINGS

FIG. 1 schematically illustrates a mammography system according to the invention.

FIG. 2 schematically illustrates a further embodiment of the mammography system according to the present invention.

FIG. 3 schematically illustrates another embodiment of the mammography system according to the present invention.

### DESCRIPTION OF THE PREFERRED EMBODIMENTS

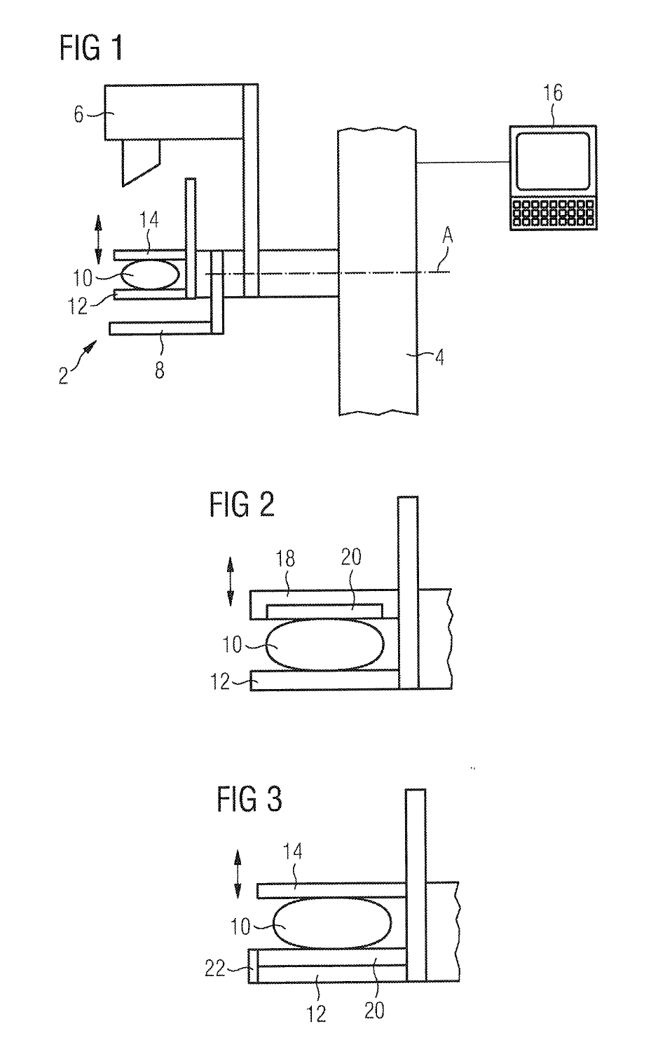

FIG. 1 shows an example of a mammography system 2 that is suitable for both a radiographic examination and a sonographic examination. An x-ray tube 6 and a detector 8 are attached to a vertical column 4 such that they can rotate around an axis A. For the examination of a breast 10, this is placed on a support plate (likewise attached to the vertical column 4) and compressed with the use of a compression plate 14 that can be moved relative to a support plate 12. Deviating from the depiction in FIG. 1, the support plate 1 can be part of the detector housing. In such mammography systems the cover plate of the detector simultaneously serves as a support plate 12. The breast 10 fixed between the support plate 12 and the compression plate 14 is located in the beam path between the x-ray tube 6 and the detector 8. To acquire a mammogram, the breast 10 in its compressed state is radiographically exposed. The mammogram acquired with the use of the detector 8 can be displayed or additionally processed with the use of a processing unit 16, for example presented via a monitor and a keyboard.

The mammography system 2 shown in FIG. 1 is likewise suitable for the implementation of tomosynthetic examinations in which 3D mammography images are acquired. For example, to acquire such 3D images a plurality of projection images of the compressed breast 10 are acquired via a rotation of the x-ray tube 6 and the detector 8 around the axis A. As is generally known, the images are processed into a 3D mammogram with the aid of the processing unit 16. Deviating from the depiction in FIG. 1, the mammography system 2 can also be designed such that the detector 8 including the support plate 12 are mounted fixed to the vertical column 4. In such a mammography system only the x-ray tube 6 is moved to acquire different projections.

Aside from radiographic examinations, the mammography system 2 is likewise provided for sonographic examination of the breast 10. The radiographic examination is thereby normally conducted chronologically before the sonographic examination. This has the advantage that, within the scope of the radiographic examination, a density of the breast can be determined using which it can be differentiated whether a subsequent sonographic examination is reasonable or not. It is likewise possible to implement the sonographic examination before the radiographic examination. In such a case, the subsequent radiographic examination can possibly be foregone, which reduces the radiation exposure of the patient. In the following the case in which the radio examination is implemented before the sonographic examination is assumed as an example. Before the actual sonographic examination (after the mammography exposure has occurred), the breast 10 is initially unloaded by raising the compression plate 14. The breast 10 thereby remains essentially at the location on the support plate 12 at which this was positioned at the beginning of the mammography examination. The compression plate 14 is now exchanged for an additional compression plate 18 and the breast 10 is recompressed. In the event that the breast does not remain on the support plate 12, it is repositioned before the sonographic examination, advantageously by the same person, advantageously a medical specialist.

FIG. 2 shows a detailed view of the mammography system 2 retrofitted in this manner for the sonographic examination, in the region of its support plate 12 and compression plate 18. The additional compression plate 18 has an ultrasound transducer 20 with which a sonogram of the breast 10 is acquired in the following. The ultrasound transducer 20 is inserted into the side of the additional compression plate 18 that faces the breast 10. The ultrasound transducer 20 is advantageously operated in a frequency range between 5 MHz and 15 MHz, additionally advantageously in a frequency range between 7 MHz and 9 MHz. The cited ultrasound frequencies are particularly suitable for the examination of breast tissue. The large-area ultrasound transducer 20 is inserted into the side the additional compression plate 18 that faces the breast 10 and is in direct contact with the compressed breast 10. Typical coupling media—for example a gel—can be used for improved injection of the ultrasound into the breast tissue.

The ultrasound transducer 20 is formed by a number of capacitively controlled micro-ultrasound transducer elements (CMUTs) arranged in the form of a matrix. Such a large-area ultrasound transducer 20 allows an examination of the breast 10 without the scanning movements that are otherwise typical in ultrasound examinations. The examination device 2 can in particular be an already existing mammography system which is made suitable for the sonographic examination by exchanging the compression plate 14. The sonographic images acquired with the aid of the ultrasound transducer 20 are displayed and possibly additionally processed with the aid of the processing unit 16.

Mammograms and sonograms or, respectively, their digital image data sets can be registered with one another with the aid of the processing unit 16. This allows a simple association of the corresponding image regions of the mammogram or, respectively, sonogram. A registration of the mammogram and the sonogram is naturally likewise possible in the case in which the mammogram is a tomosynthetic 3D mammogram. The registration is significantly simplified (if not enabled in the first place) by the very similar bearing of the breast 10 in the sonographic examination and the radiographic examination.

FIG. 3 shows a further exemplary embodiment according to which the mammography system 2 that is generally described in connection with FIG. 1 was retrofitted such that the ultrasound transducer 20 is now present on the underside of the breast 10 between the support plate 12 and the breast 10. The ultrasound transducer 20 can be accommodated by a guide rail 22 connected with the support plate 12. The ultrasound transducer 20 can be arranged in the same manner on the top side of the breast 10, thus between the compression plate 14 and the breast 10. In each case it is advantageous if the ultrasound transducer 20 is brought from a direction perpendicular to the plane of the paper into its desired position. A patient (who normally stands more or less frontally in front of the mammography system 2—see FIG. 1—for examination of the breast 10 can in this case remain in the posture that the patient has adopted for the radiographic examination, even during the insertion of the ultrasound transducer 20. If the ultrasound transducer 20 is positioned on the side of the support plate 12 that faces the breast 10, to insert the ultrasound transducer 20 it is merely necessary to raise the breast 10 slightly. If the ultrasound transducer 20 is arranged on the side of the compression plate 18 that faces the breast 10, after a radiographic examination has occurred the compression plate 18 is raised briefly with unloading of the breast 10, the ultrasound transducer 20 is inserted (possibly directed via a corresponding mount 22), and the breast 10 is recompressed between the ultrasound transducer 20 and the support plate 12. In each case the breast is advantageously positioned by the same person, for example a medical technician.

Although modifications and changes may be suggested by those skilled in the art, it is the intention of the inventors to embody within the patent warranted heron all changes and modifications as reasonably and properly come within the scope of their contribution to the art.

1.-17. (canceled)

18. a method to examine a breast comprising the steps of:
positioning a breast on a support plate of a mammography system and compressing the breast between said support plate and a compression plate;
irradiating the breast compressed between the support plate and the compression plate to obtain a radiographic mammogram of the compressed breast;
compressing the breast between an ultrasound transducer, comprising a plurality of ultrasound transducer elements arranged in a plane, and one of said compression plate or said support plate, with said ultrasound transducer in direct contact with the breast; and
irradiating the breast with ultrasound from said ultrasound transducer to obtain a sonogram of the breast compressed between said ultrasound transducer and said one of said compression plate or said support plate.

19. A method as claimed in claim 18 wherein said compression plate is a first compression plate, and comprising compressing the breast between said ultrasound transducer and said one of said compression plate or said support plate comprises exchanging said first compression plate for a second compression plate in which said ultrasound transducer is located.

20. A method as claimed in claim 18 wherein said support plate is a first support plate, and comprising compressing the breast between said ultrasound transducer and said one of said compression plate or said support plate by exchanging said first support plate for a second support plate in which said ultrasound transducer is located.

21. A method as claimed in claim 18 comprising compressing the breast between said ultrasound transducer and said one of said compression plate or said support plate by temporarily mounting said ultrasound transducer at a side of said compression plate that faces the breast to obtain said sonogram, and compressing the breast between said compression plate with said ultrasound transducer mounted thereat, and said support plate.

22. A method as claimed in claim 21 comprising temporarily mounting said ultrasound transducer at said side of said compression plate by sliding said ultrasound transducer into said side of said compression plate while said side of said compression plate remains facing the breast.

23. A method as claimed in claim 18 comprising compressing the breast between said ultrasound transducer and said one of said compression plate or said support plate by temporarily mounting said ultrasound transducer at a side of said support plate that faces the breast while obtaining said sonogram, and compressing the breast between said support plate with said ultrasound transducer mounted thereat, and said compression plate.

24. A method as claimed in claim 23 comprising temporarily mounting said ultrasound transducer at said side of said support plate by sliding said ultrasound transducer into said side of said support plate while said side of said support plate remains facing the breast.

25. A method as claimed in claim 21 comprising, in a processor, bringing an image data set representing said radiographic mammogram into registration with an image data set representing said sonogram.

26. A method as claimed in claim 18 comprising generating said radiographic mammogram by irradiating the breast compressed between said compression plate and said support plate from a plurality of different exposure directions, to obtain a plurality of x-ray images respectively from said different exposure directions, and reconstructing a tomosynthetic 3D mammogram from said plurality of x-ray images.

27. A method as claimed in claim 26 comprising, in a processor, bringing a data set representing said tomosynthetic 3D mammogram into registration with a data set representing said sonogram.

28. A method as claimed in claim 18 comprising operating said ultrasound transducer at a frequency in a range between 5 MHz and 15 MHz to produce said sonogram.

29. A method as claimed in claim 18 comprising operating said ultrasound transducer at a frequency in a range between 7 mHz and 9 mHz to produce said sonogram.

30. A mammography system comprising:
an x-ray imaging system;
a compression plate and a support plate adapted to compress a breast therebetween to obtain a radiographic exposure of the breast by operating said x-ray imaging system;
a replacement plate that temporarily replaces one of said compression plate or said support plate, said replacement plate containing an ultrasound transducer at a side thereof facing toward the breast, said ultrasound transducer comprising a plurality of transducer elements arranged in a plane; and
said replacement plate compressing the breast between the replacement plate and the other of said compression plate or said support plate that is not replaced by said replacement plate, to obtain a sonogram of the breast by irradiating the breast with ultrasound from said ultrasound transducer.

31. A mammography system as claimed in claim 30 wherein said ultrasound transducer is located at a side of said replacement plate that faces the breast to obtain said sonogram.

32. A mammography system as claimed in claim 30 wherein said ultrasound transducer comprises a plurality of capacitively controlled micro-ultrasound transducer elements.

33. A mammography system comprising:
an x-ray imaging system;
a support plate and a compression plate adapted to compress a breast therebetween to obtain a radiographic mammogram of the breast with said x-ray imaging system;
one of said support plate or said compression plate comprising a mount at a side thereof facing the breast;
an ultrasound transducer configured to be removably fitted into and held in said mount, said one of said compression plate or said support plate with said ultrasound transducer fitted therein being adapted to compress the breast against the other of said support plate or said compression plate to obtain a sonogram of the breast by irradiating the breast with ultrasound from the ultrasound transducer.

34. A mammography system as claimed in claim 33 wherein said mount allows sliding of said ultrasound transducer into said mount to face the breast.

35. A mammography system as claimed in claim 33 wherein said ultrasound transducer comprises a plurality of capacitively controlled micro-ultrasound transducer elements.
